(19) United States
(12) Patent Application Publication
(10) Pub. No.: US 2014/0127202 A1
(43) Pub. Date: May 8, 2014

(54) ANTITUMOUR COMBINATIONS CONTAINING A VEGF INHIBITING AGENT AND IRINOTECAN

(71) Applicant: Aventis Pharma S.A., Antony (FR)

(72) Inventors: Marie-Christine BISSERY, Charenton le Pont (FR); Marielle CHIRON-BLONDEL, Paris (FR); Pascale LEJEUNE, Vitry (FR); Patricia VRIGNAUD, Combs la Ville (FR)

(73) Assignee: Aventis Pharma S.A., Antony (FR)

(21) Appl. No.: 14/153,795

(22) Filed: Jan. 13, 2014

Related U.S. Application Data
(63) Continuation of application No. 12/651,767, filed on Jan. 4, 2010, now abandoned.
Application No. 12/651,767 is a continuation of application No. PCT/FR2008/000943, filed on Jul. 2, 2008.

(30) Foreign Application Priority Data
Jul. 5, 2007 (FR) 0704868

(51) Int. Cl. A61K 39/395; A61K 31/4745

(52) U.S. Cl. CPC A61K 39/3955; A61K 31/4745
USPC 424/134.1

(57) ABSTRACT

Disclosed are antitumor combinations of VEGF inhibitors with Irinotecan and the use thereof in the treatment of neoplastic diseases.

The present invention relates to the combinations of a VEGF inhibitor and of a chemotoxic agent of the topoisomerase inhibitor class, useful in the treatment of neoplastic diseases.

VEGF inhibitors, which are inhibitors of vascular endothelial growth factor, are in the majority of cases biological products selected from soluble receptors, antisenses, RNA aptamers and antibodies. The topoisomerase inhibitors of use in the treatment of known neoplastic diseases are selected from camptothecins, including CPT 11, topotecan and pyridobenzoindole. The present combination is in particular directed toward the treatment of colon cancer or of stomach cancer.

The description and the preparation of the VEGF inhibitor preferably used in the invention, which is a VEGF Trap chimeric protein, is described in patent application WO 00/75319. There are several embodiments of the chimeric protein.

The embodiment corresponding to VEGF Trap is the one described in FIG. 24 (sequence) of WO 00/75319. The VEGF Trap used in the invention is a fusion protein comprising the signal sequence of VEGFR1 fused to the D2 Ig domain of the VEGFR1 receptor, itself fused to the D3 Ig domain of the VEGFR2 receptor, in turn fused to the Fc domain of IgG1, also known as VEGFR1R2-FcΔC1 or Flt1D2.Flk1D3.FcΔC1.

In general, the doses of VEGF Trap used in humans, which depend on factors specific to the individual to be treated, are between 1 and 10 milligrams per kilo when the administration is carried out subcutaneously or intravenously.

Among the topoisomerase inhibitors, irinotecan, also known under the nonproprietary name CPT-11, is preferably used.

Irinotecan is generally used intravenously at a dose of between 100 mg/m² and 500 mg/m² depending on the administration scheme. A dose of 150 mg/m² is, for example, used for a weekly scheme and a dose of between 200 and 400 mg/m² is, for example, used for a scheme every three weeks.

An article by H Hurwitz, L Fehrenbacher, W Novotny, T Cartwright, J Hainsworth, W Heim, J Berlin, A Baron, S Griffing, E Holmgren, N Ferrara, G Fyfe, B Rogers, R Ross, F Kabbinavar, published in “The New England Journal of Medicine” has described a clinical trial proving a better survival rate when the combination of bevacizumab with irinotecan, 5FU and leucovorin is used, compared with the same combination not containing bevacizumab. In this clinical trial, there is nothing to prove that the improvement in the survival rate comes from the combination of irinotecan with bevacizumab, it may just as easily come from the combination of 5FU or of leucovorin with bevacizumab, or may come from the quadruple combination. Now, since it is known that each of the anticancer agents brings toxic side effects, along with its therapeutic effect, it appears to be advisable to limit their presence as much as possible, especially when the same effect can be obtained in the absence of at least one of them. Furthermore, this article does not provide evidence of any synergistic effect within the meaning of Corbett, i.e. an effect which cannot be obtained with each of the elements of the combination used alone at its maximum tolerated dose.

VEGF Trap is a soluble receptor created by fusion of the second Ig domain of VEGFR-1 with the third Ig domain of VEGFR-2, which is subsequently fused to the Fc part of a human IgG1. Like the VEGFR-1 receptor, aflibercept (VEGF Trap) has a very high affinity for VEGF-A, with a Kd of 0.5 picoM. The high-affinity binding of VEGF Trap with VEGF-A results in the formation of a complex which prevents VEGF from binding to and activating its receptors at the surface of cells.

In comparison with Avastin (or bevacizumab), VEGF Trap is a soluble receptor, whereas Avastin is an antibody directed against VEGF-A. VEGF Trap has a much higher affinity for VEGF-A than that of Avastin, and a different selectivity profile since VEGF Trap also binds to the other ligands of VEGFR1-2 receptors, i.e. to PIGF (placental growth factor) and to VEGF-B. Furthermore, VEGF Trap has a molecular weight which is substantially less than that of Avastin (115 kDa for aflibercept versus 160 kDa for Avastin), more favorable to penetration in solid tumors.

It has now been found, and it is this which is the subject of the present invention, that the effectiveness of VEGF inhibitors can be considerably improved when they are administered in combination with at least one substance of therapeutic use in anticancer treatments which has a mechanism of action different than that of VEGF inhibitors.

Moreover, since the activity of the products depends on the doses used, it is possible to use higher doses and to increase the activity by reducing the toxicity phenomena or by delaying their appearance, through the combining with the VEGF inhibitors or with their analogs of other therapeutically active substances of growth factors of hematopoietic type, such as G-CSF or GM-CSF, or certain interleukins.

More particularly, the invention relates to the combinations of VEGF Trap with irinotecan.

The improved effectiveness of a combination according to the invention can be demonstrated by determining the therapeutic synergism.

A combination shows a therapeutic synergism if it is therapeutically superior to both the constituents used at the optimum dose thereof.

In order to demonstrate the effectiveness of a combination, it may be necessary to compare the maximum tolerated dose of the combination with the maximum tolerated dose of each of the isolated constituents in the study under consideration. This effectiveness can be quantified, for example, by the log₁₀ cell kill, which is determined according to the following equation:

log₁₀ cell kill=T-C (days)/3.34×T_{d}

in which T-C represents the delay in growth of the cells, which is the average time, in days, for the tumors of the treated group (T) and the tumors of the control group (C) to have reached a predetermined value (1 g for example), and T_{d} represents the time, in days, necessary for the volume of the tumor to double in the control animals [T. H. Corbett et al., Cancer, 40, 2660.2680 (1977); F. M. Schabel et al., Cancer Drug Development, Part B, Methods in Cancer Research, 17, 3-51, New York, Academic Press Inc. (1979)]. A product is considered to be active if log₁₀ cell kill is greater than or equal to 0.7. A product is considered to be very active if log₁₀ cell kill is greater than 2.8.

The combination, used at its own maximum tolerated dose, in which each of the constituents is present at a dose generally less than or equal to its maximum tolerated dose, will show therapeutic synergy when the log₁₀ cell kill is greater than the value of the log₁₀ cell kill of the best constituent when it is administered alone, and in particular has a superiority of at least one log cell kill.

The effectiveness of the combinations on solid tumors can be determined experimentally in the following way:

The animals subjected to the experiment, generally mice, are grafted bilaterally, subcutaneously, with 30 to 60 mg of an HCT116 human tumor fragment (Brattain, M. G., Fine, W. D., Khaled, F. M., Thompson, J. and Brattain, D. E., Heterogeneity of malignant cells from a human colonic carcinoma. Cancer Res., 1981, 41, 1751-1756) on day 0. The animals bearing the tumors are randomized before being subjected to the various treatments and controls. In the case of treatment of tumors of the present invention, the tumors were allowed to develop to a size of between 48 and 294 mg, which made it possible to have a median tumor per group of between 129 and 162 mg. The animals which underwent the treatment with VEGF Trap alone had a weight of between 17.1 and 22.7 g, the animals having undergone the treatment with irinotecan alone had a weight of between 17.5 and 22.3 g, and those which received the combination had a weight of between 17.5 and 23.6 g. Animals bearing tumors were also subjected to the same treatments with the excipient alone in order to be able to dissociate the toxic effect of the excipient from the actual effect of the chemotherapy on the tumor. The chemotherapy was begun on day 12 after the tumor graft. The VEGF Trap injections were given subcutaneously simultaneously with the irinotecan injections, which themselves were given intravenously, according to a daily double injection. These injections were carried out on days 12, 15 and 18 after implantation of the tumor. The various groups of animals are weighed three to four times per week until the maximum weight loss is reached, and then the groups are weighed at least once a week until the end of the trial.

The tumors are measured two or three times a week until the tumor reaches approximately 2 g or until the death of the animal if the latter occurs before the tumor reaches 2 g. The animals are autopsied at the time of sacrifice.

The antitumor activity is determined according to the various parameters recorded.

By way of examples, the following tables give the results obtained with combinations of VEGF Trap and of irinotecan used at their optimum dose.

The present invention also relates to the kits of pharmaceutical compositions containing the products used in the combinations according to the invention.

The products which constitute the combination may be administered simultaneously, separately or sequentially in such a way as to obtain the maximum effectiveness of the combination; it being possible for each administration to have a variable duration ranging from a rapid total administration to a continuous infusion.

As a result of this, for the purpose of the present invention, the combinations are not only limited to those which are obtained by physical association of the constituents, but also to those which allow a separate administration that can be simultaneous or sequential.

The compositions according to the invention are preferably compositions that can be administered parenterally.

Compositions for parenteral administration are generally pharmaceutically acceptable sterile solutions or suspensions which may optionally be prepared extemporaneously at the time of use. For the preparation of nonaqueous solutions or suspensions, natural plant oils such as olive oil, sesame oil or liquid paraffin, or injectable organic esters such as ethyl oleate, may be used. Aqueous sterile solutions may be constituted of a solution of the product in water. Aqueous solutions are suitable for intravenous administration insofar as the pH is suitably adjusted and the isotonicity is produced, for example, by means of a sufficient amount of sodium chloride or of glucose. The sterilization can be carried out by heating or by any other means which does not detrimentally alter the composition. The combinations may also be in the form of liposomes or in the form of an association with supports such as cyclodextrins or polyethylene glycols.

In the combinations according to the invention, the application of the constituents of which may be simultaneous, separate or sequential, it is particularly advantageous for the amount of VEGF Trap derivative to represent from 10% to 80% by weight of the combination, it being possible for this content to vary according to the nature of the associated substance, to the desired effectiveness and to the nature of the cancer to be treated.

The combinations according to the invention are of particular use in the treatment of colon cancer and/or stomach cancer. In particular, they can have the advantage of being able to use the constituents at doses which are much lower than those at which they are used alone.

The following example illustrates a combination according to the invention.

### EXAMPLE

Vials of 1 cm³ containing 25 mg of VEGF Trap which are diluted in a buffer of 5 mM phosphate, 5 mM sodium citrate, 100 mM sodium chloride, polysorbate 20 and 20% sucrose are prepared according to the usual technique, for subcutaneous administration. The administration volume per mouse is 0.1 ml. The VEGF Trap is administered once a day, on days 12, 15 and 18 after implantation of the tumor.

0.3 ml per mouse is prepared, according to the usual technique, for intravenous administration, from a commercially available solution at 20 mg/ml of irinotecan to be diluted with 5% dextrose in water.

These solutions are administered simultaneously, after a suitable dilution.

The treatment with irinotecan is repeated twice a day, with a 4-hour interval, on days 12, 15 and 18 after implantation of the tumor.

The results of the trial are attached in the appended table.

Tumor doubling time=3.2 days.

Abbreviations used: (T-C) delay in growth of the tumor, Ick=log cell kill.

Toxicity was observed for irinotecan alone at the doses of 52.4, 32.5 and 20.2 mg/kg/injection owing to a death at the dosage of 52.4 and a weight loss of greater than 20% for the two lower dosages. Thus, the maximum tolerated dose for irinotecan was 12.5 mg/kg/inj (total injected dose of 75.0 mg/kg). The dose of 12.5 mg/kg/injection was found to be active with an Ick of 1.8.

For the VEGF Trap, the product was well-tolerated at all the dosages tested and was found to be active with an Ick of 1.7 at 40 mg/kg/administration and 25 mg/kg/administration. The lower dose of 10 mg/kg/administration is also active, with an Ick of 1.3. The dose of 2.5 mg/kg/administration is inactive.

For the combination of irinotecan at 32.5 mg/kg/inj, whatever the dose of VEGF Trap, the combination was found to be toxic with a weight loss of 18% close to toxicity. The lower dose of 20.2 mg/kg/inj of irinotecan with 40 mg/kg of VEGF Trap was considered to be the maximum tolerated dose. This dose had an Ick of 3.0, judged to be very active. The same level of activity was found with the lower doses of VEGF Trap, such as 25, 10, 2.5 mg/kg/administration (Ick of 2.9, 3.0 and 2.9, respectively).

Irinotecan at 12.5 mg/kg/inj combined with VEGF Trap at 40 mg/kg/administration is active with an Ick of 2.7. This antitumor activity is maintained with 25 and 10 mg/kg of VEGF Trap (Ick of 2.9 and 2.7, respectively). The combination with 2.5 mg/kg/administration of VEGF Trap has an activity of 2.0 Ick.

In conclusion, the activity of the combination of VEGF Trap with irinotecan shows a synergistic effect with a log cell kill of 3.0, at the maximum tolerated dose of the combination, which corresponds to more than 1 log cell kill compared with the activity of each of the compounds used alone, which exhibits a log cell kill of 1.8 and 1.7 (for irinotecan at 12.5 mg/kg/injection and VEGF Trap at 40 mg/kg/administration, respectively). An antitumor activity is maintained at several levels of doses below the maximum tolerated dose of the combination.

| | | | | | | Death | Weight | Average | | | | |
|---|---|---|---|---|---|---|---|---|---|---|---|---|
| | | | | | | due to | loss of | time for | | | | |
| | | | | | | the | the | the | | | | |
| | | | Dosage | | Total | product, | mouse | tumor to | | | Survivors | |
| | | Route of | in mg/kg | | dose | with | (date of | reach | | | without | |
| | Agent | admin- | per | Scheme | in | day of | low- | 750 mg | T-C in | Log | tumor at | |
| Group | (alone) | istration | injection | in days | mg/kg | death | point) | in days | days | cell kill | day 144 | Comments |
| 6 | CPT-11 | i.v. | 52.4 | 12, 15, 18 | 314.4 | 1/8 (23) | −27.0 | — | — | — | — | Toxic |
| | | 0.3 ml | | | | | (21) | | | | | |
| 7 | | | 32.5 | (2x/d) | 195.0 | 0/7 | −23.1 | — | — | — | — | Toxic |
| | | | | | | | (19) | | | | | |
| 8 | | | 20.2 | (4 h apart) | 121.2 | 0/8 | −21.9 | — | — | — | — | Toxic |
| | | | | | | | (19) | | | | | |
| 9 | | | 12.5 | | 75.0 | 0/8 | −15.9 | 37.6 | 19.1 | 1.8 | 0/8 | HNTD |
| | | | | | | | (21) | | | | | active |
| 2 | VEGF | s.c. | 40.0 | 12, 15, 18 | 120.0 | 0/8 | −3.1 (13) | 36.8 | 18.3 | 1.7 | 0/8 | HDT |
| | Trap | 0.1 ml | | | | | | | | | | active |
| 3 | | | 25.0 | | 75.0 | 0/8 | −2.2 (13) | 36.8 | 18.3 | 1.7 | 0/8 | active |
| 4 | | | 10.0 | | 30.0 | 0/8 | −5.0 (19) | 32.0 | 13.5 | 1.3 | 1/8 | active |
| 5 | | | 2.5 | | 7.5 | 0/8 | −8.3 (19) | 20.2 | 1.7 | 0.2 | 0/8 | inactive |
| 10 | CPT-11 | i.v. | 32.5 | 12, 15, 18 | 195.0 | 0/8^{b} | −16.2 | — | — | — | — | Toxic |
| | | 0.3 ml | | (2x/d) | | | (21) | | | | | |
| | VEGF | s.c. | 40.0 | 12, 15, 18 | 120.0 | | | | | | | |
| | Trap | 0.1 ml | | | | | | | | | | |
| 11 | | | 32.5 | | 195.0 | 0/8 | −18.6 | — | — | — | — | Toxic |
| | | | 25.0 | | 75.0 | | (21) | | | | | |
| 12 | | | 32.5 | | 195.0 | 0/8 | −18.2 | — | — | — | — | Toxic |
| | | | 10.0 | | 30.0 | | (19) | | | | | |
| 13 | | | 32.5 | | 195.0 | 0/8 | −18.4 | — | — | — | — | Toxic |
| | | | 2.5 | | 7.5 | | (19) | | | | | |
| 14 | | | 20.2 | | 121.2 | 0/8 | −13.0 | 50.9 | 32.4 | 3.0 | 0/8 | HNTD |
| | | | | | | | (21) | | | | | highly active |
| | | | 40.0 | | 120.0 | | | | | | | |
| 15 | | | 20.2 | | 121.2 | 0/8 | −11.9 | 49.6 | 31.1 | 2.9 | 0/8 | high active |
| | | | 25.0 | | 75.0 | | (19) | | | | | |
| 16 | | | 20.2 | | 121.2 | 0/8 | −13.0 | 50.7 | 32.2 | 3.0 | 0/8 | high active |
| | | | 10.0 | | 30.0 | | (19) | | | | | |
| 17 | | | 20.2 | | 121.2 | 0/8 | −12.5 | 49.7 | 31.2 | 2.9 | 0/8 | high active |
| | | | 2.5 | | 7.5 | | (19) | | | | | |
| 18 | | | 12.5 | | 75.0 | 0/8 | −11.0 | 46.8 | 28.3 | 2.7 | 0/8 | active |
| | | | 40.0 | | 120.0 | | (19) | | | | | |
| 19 | | | 12.5 | | 75.0 | 0/8 | −11.9 | 49.8 | 31.3 | 2.9 | 0/8 | high active |
| | | | 25.0 | | 75.0 | | (19) | | | | | |
| 20 | | | 12.5 | | 75.0 | 0/8 | −10.0 | 47.0 | 28.5 | 2.7 | 0/8 | active |
| | | | 10.0 | | 30.0 | | (21) | | | | | |
| 21 | | | 12.5 | | 75.0 | 0/8 | −14.3 | 40.0 | 21.5 | 2.0 | 0/8 | active |
| | | | 2.5 | | 7.5 | | (21) | | | | | |
| 1 | Vehicle | s.c. | — | 12, 15, 18 | | | −9.6 (25) | 18.5 | | | 0/10 | |
| | | 0.1 ml | | | | | | | | | | |
| | | i.v. | — | 12, 15, 18 | | | | | | | | |
| | | 0.3 ml | | (2x/d) (4 h | | | | | | | | |
| | | | | later) | | | | | | | | |

What is claimed is:

1. A method of treating a neoplastic disease comprising administering to a patient in need thereof a combination of a VEGF inhibitor with irinotecan.

2. The method according to claim 1, wherein the VEGF inhibitor is a VEGF Trap.

3. The combination according to claim 2, wherein the amount of VEGF Trap represents from 10% to 80% by weight of the combination.

4. The method according to claim 1, wherein said method does not include any other chemotoxic derivative having a therapeutically synergistic effect in the treatment of neoplastic diseases.

5. A product comprising a VEGF inhibitor and irinotecan, as a combined preparation for simultaneous, separate or sequential use in anticancer therapy.

6. The product according to claim 5, wherein the VEGF inhibitor is a VEGF Trap.

7. The product according to claim 6, wherein the amount of VEGF Trap represents from 10% to 80% by weight of the combined weight of VEGF Trap and irinotecan.

8. The product according to claim 5 which does not include any other chemotoxic derivative having a therapeutically synergistic effect in the treatment of neoplastic diseases.

9. A combination containing a VEGF inhibitor with irinotecan.

10. The combination according to claim 9, containing a VEGF Trap with irinotecan.

11. The combination according to claim 10, containing from 10% to 80% by weight of VEGF Trap.
